(19) United States
(12) Patent Application Publication
(10) Pub. No.: US 2021/0316143 A1
(43) Pub. Date: Oct. 14, 2021

(54) PERIPHERAL NERVE MODULATOR AND METHODS RELATING TO PERIPHERAL NERVE MODULATION

(71) Applicant: University of Florida Research Foundation, Inc., Gainesville, FL (US)

(72) Inventors: Alexander DRU, Gainesville, FL (US); Kyle Douglas ALLEN, Gainesville, FL (US); Kevin OTTO, Gainesville, FL (US); Lauren Savannah DEWBERRY, Gainesville, FL (US)

(21) Appl. No.: 17/274,027

(22) PCT Filed: Sep. 6, 2019
(86) PCT No.: PCT/US2019/049994

§ 371 Date: Mar. 5, 2021

Related U.S. Application Data
(60) Provisional application No. 62/728,224, filed on Sep. 7, 2018.

(51) Int. Cl. A61N 1/36; A61N 1/05

(52) U.S. Cl. CPC A61N 1/36071; A61N 1/36171; A61N 1/36135; A61N 1/0558

(57) ABSTRACT

Described herein are peripheral nerve modulators (i.e. neuromodulators) and methods relating to peripheral nerve modulation. In an embodiments of peripheral neurostimulators described herein, neuromodulators comprise: a neuromodulator comprising a power source and an electric pulse generator; a solenoidal lead comprising a flexible polymer and a plurality of metal contacts, and a cable physically connecting the solenoidal lead to the neuromodulator.

### CROSS-REFERENCE TO RELATED APPLICATION

This application claims priority to co-pending U.S. provisional patent application entitled “PERIPHERAL NERVE MODULATOR AND METHODS RELATING TO PERIPHERAL NERVE MODULATION”, having Ser. No. 62/728,224, filed on Sep. 7, 2018, which is entirely incorporated herein by reference.

### BACKGROUND

There are currently no implantable peripheral nerve pain modulating systems on the market capable of kilohertz stimulation. Pain modulation in neurosurgery is focused on implantable spinal cord simulators to block ascending pain signals. The implantation of these devices currently requires invasive thoracic multilevel laminectomies which put the spinal cord at iatrogenic risk for damage if there was a complication with surgery. Damage to the spinal cord at this level could cause bilateral lower extremity paresis or plegia, and/or loss of bowel/bladder/sexual function. Accordingly, there is a need to address the aforementioned deficiencies and inadequacies.

### SUMMARY

Described herein are peripheral nerve modulators and methods relating to peripheral nerve modulation. In an embodiments of peripheral neurostimulators described herein, neuromodulators comprise: a neuromodulator comprising a power source and an electric pulse generator; a solenoidal lead comprising a flexible polymer and a plurality of metal contacts, and a cable physically connecting the solenoidal lead to the neuromodulator. The cable can comprise a plurality of contact leads providing electrical communication between metal contacts of the solenoidal lead and the neurostimulator. In certain aspects, the solenoidal lead contains a plurality of metal contacts, each of the plurality of metal contacts are in electrical communication with the neuromodulator through a contact lead, wherein none of the metal contacts shares a contact lead with another metal contact, wherein the metal contacts are configured to make contact with a peripheral nerve of a subject from an inner surface of the solenoidal lead, the metal contacts extending equidistantly the length of a longitudinal axis of the solenoidal lead.

Also described herein are methods of peripheral nerve modulation. In certain embodiments, a method can be a method of treatment of a disease or symptoms of a disease in a subject. The method can comprise providing a subject in need thereof; implanting a neurostimulator as described herein (the neuromodulator having a nerve cuff or solenoidal lead) into a subject in need thereof; and delivering kilohertz stimulation to the subject in need thereof from the neurostimulator.

Also described herein are embodiments of solenoidal leads. In an embodiment, a solenoidal lead comprises: a flexible polymer, a plurality of contact leads, and a plurality of metal contacts, the plurality of contact leads and metal contacts partially encased in the flexible polymer. In certain aspects, each of the plurality of metal contacts is in contact with a contact lead. In certain aspects, none of the metal contacts share a contact lead with another metal contact. In certain aspects, a surface of each of the metal contacts is configured to make contact with a peripheral nerve of a subject from an inner surface of the solenoidal lead, the metal contacts extending equidistantly the length of a longitudinal axis of the solenoidal lead.

In an embodiment, the solenoidal lead further comprises a cable encasing portions of the contact leads not encased in the flexible polymer. In an embodiment, the solenoidal lead is helically shaped. In an embodiment, the solenoidal lead is configured to wrap around a peripheral nerve prior to implantation in a subject. In an embodiment, the solenoidal lead comprises about 3 turns around the nerve or more. In an embodiment, the solenoidal lead comprises about 3 to about 5 turns around the nerve or more. In an embodiment, the solenoidal lead comprises at least 4 metal contacts per turn of the solenoidal lead. In an embodiment, the plurality of contact leads and metal contacts are platinum. In an embodiment, the distance between turns is about 0.5 cm. In an embodiment, the contacts are platinum-iridium contacts.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A and 1B depict an embodiment of a peripheral nerve modulator as described herein. According to the embodiment depicted, the peripheral nerve modulator comprises a pulse generator 205 that is connected to a solenoid lead 201 by way of a cable 203, the solenoid lead being configured to wrap around and make contact with the outside diameter of a peripheral nerve 101 along a longitudinal axis of the peripheral nerve in the direction A. FIG. 1B illustrates a perspective view of an embodiment of the solenoidal lead of FIG. 1.

FIG. 2 demonstrates data of a Von Frey nociception assay in a rodent model of neuropathy/allodynia with and without kilohertz stimulation.

FIGS. 3 and 4 illustrate recordings of stimulated compound action potentials in a live rat sciatic nerve (average of 20 stimulations per line) with the bipolar stimulation cuff around the sciatic nerve and the recording cuff more distally around the tibial nerve (right side of the animal).

FIGS. 5A-5M are photographs illustrating a reduced-to-practice embodiment of how to perform the surgical model and implant aspects of the neurostimulation system described in the present disclosure.

FIGS. 6A-6B show an embodiment of positioning on a sciatic nerve (FIG. 6A) and relative positioning of aspects of an embodiment of a neurostimulation system (FIG. 6B) as described herein.

FIG. 7 demonstrates data of a Von Frey nociception assay in a first cohort (N=5) of an acute rodent model of neuropathy/allodynia with and without kilohertz stimulation.

FIG. 8 demonstrates data of a Von Frey nociception assay in a second cohort (N=6) of rodent model of a chronic neuropathy/allodynia with and without kilohertz stimulation.

FIG. 9 is an amplitude-time graph showing compound action potentials (CAPs) following sciatic nerve stimulation with integrals of peaks corresponding to specific fiber activation.

FIG. 10 illustrates plots of rank-order gait data of rat gait (following sciatic crush surgery with and without stimulation scored by blinded researchers

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of neurosurgery, neurology, electrical engineering, and mechanical engineering.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

Unless otherwise defined, all technical and scientific terms used herein have the same meaning as commonly understood by one of ordinary skill in the art of molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the term “about” can mean −20% to +20%, −15% to +15%, −10% to +10%, −5% to +5%, and the like.

As used herein, “partial” encasement of the metal contacts means that one side is not encased in the flexible polymer and exposed to the nerve of a subject. “Partial” encasement of the leads means a length of the leads from an end at the metal contact is encased, which another longer length is not encased in the flexible polymer.

As used herein, a “subject” can be a mammal or human experiencing peripheral nerve dysfunction, or any one or more of the symptoms of peripheral nerve dysfunction described in the following section.

### Discussion

Systems and methods as described herein utilize implantable devices at the level of one or more peripheral nerves for the purpose of neuromodulation. By controlling peripheral nerve pain at the level of the peripheral nerve using implantable devices, rather than implanting at the level of the spinal cord, the resulting surgery would cause a fraction of the blood loss (leading implantation to be tolerated by a wider patient population), and the risk profile of the surgery would be less in the event of a catastrophic complication (with the most reasonable worst case scenario being damage to the sciatic nerve, resulting in unilateral leg dysfunction). Systems and methods as described herein also result in more targeted therapy with a lower chance of side effects by acting peripherally rather than at the level of the spinal cord.

Embodiments of the present disclosure provide for neuromodulation devices, systems, and methods of use (i.e. variable neuromodulation) that can be implanted subcutaneously at the level of one or more peripheral nerves of interest rather than at the level of the spinal cord. It is note that in certain aspects, “neuromodulation” is used interchangeably with “neurostimulation” in the present disclosure as would be understood by one of skill in the art.

Neuromodulation devices and systems as described herein comprise or otherwise be devices which can be implanted into a subject and can modulate (stimulate, inhibit, block, etc) signals of a nerve that is associated with the device. Without intending to be limiting, examples of peripheral nerves (i.e. nerves that lie outside of the central nervous system) that can be modulated by devices and methods as described herein include: the phrenic nerve, axillary nerve, the radial nerve, the median nerve, the ulnar nerve, the intercostal nerve, the femoral nerve, the sciatic nerve, the common peroneal nerve, and the tibial nerve. Solenoidal leads as described herein can be pre-configured to wrap around nerves (i.e. axon[s] of a peripheral nerve [bundle]) as described herein, and can be configured to wrap around the nerve before implantation.

Neuromodulation devices (also referred to herein as neurostimulators) can be comprised of a pulse generator, which can comprise a power supply (for example a battery), which can be connected to a solenoidal lead by way of a cable. The solenoidal lead can be constructed of a flexible polymer with a plurality of contacts (the contacts embedded in the solenoidal lead and making contact with the nerve circumferentially around the nerve) from one or more leads that can make electrical contact with the nerve. The solenoidal lead has a helical shape, and is configured to wrap around a nerve of interest (for example the sciatic nerve) prior to implantation. In an embodiment of methods according to the present disclosure, the lead is a nerve cuff configured to deliver kilohertz stimulation. Neuromodulation devices as described herein can further comprise an internal accelerator configured to sense the motion of the subject in which it is implanted and delivery or withhold stimulation via a feedback loop and the symptoms of a particular subject.

Neurostimulation systems as described herein can comprise one or more neuromodulation devices as described herein.

In embodiments, the solenoidal lead can be a continuous helical shape pre-configured (i.e. configured prior to implantation) to encase the outer surface or circumference of a nerve. The inner diameter of the helix can be a diameter that won't cause compression of the nerve, or otherwise damage the nerve. The continuous helical shape of the lead can comprise about 3 to about 5 complete turns of the polymer or more. In an embodiment, the continuous helical shape of the lead comprises 3 complete turns of the lead around the nerve. In an embodiment, the continuous helical shape of the lead comprises 4 complete turns of the lead around the nerve. In an embodiment, the continuous helical shape of the lead comprises 3 to 5 complete turns of the lead around the nerve.

In embodiments, the solenoidal lead can comprise a plurality of square electrode contacts at different distances along the loops or turns of the helix that make electrical contact with the nerve. In certain aspects, the electrode contacts are spaced throughout the lead so that the nerve has total circumferential contact when looked at from the total contact surface of electrode squares to the nerve surface. In embodiments, there can be at least 4 contacts in contact with the nerve per turn of the helical lead. The contacts can offer discrete or circumferential multi-contact stimulation along the surface of the nerve with each contact adhered to a separate wire (or lead). This allows for some or all of the contacts to be stimulated at the same time, or pulsed differentially. In embodiments, the helical shape of the solenoidal lead is preconfigured to wrap around a nerve of known dimensions, for example the sciatic nerve, and the diameter of the helix is such that it can receive the outer circumference of the nerve while ensuring electrical contact with the contacts and without causing compression of the nerve.

Pulse generators as described herein can be an implantable pulse generator configured to produce and deliver kilohertz frequency stimulation to anatomical structures within the body (an example of which being the “Senza” pulse generator from Nevro Inc.). Pulse generators can have an internal power source. Pulse generators as described herein can be configured so that impedances and contact leads can be checked and programmed by the user or a physician by a wireless communication protocol, such as, for example, Bluetooth®.

Power sources can be a battery (for example, without intending to be limiting, a primary cell battery, lithium battery, and the like). In an embodiment, the power source can be an internal battery that is integrated with the pulse generator.

Flexible polymers (i.e. elastic polymers) which can be used for construction of the solenoid lead can be a silicone encasement or flexible polyethylene polymer. Other flexible polymers that can be incorporated into systems and devices as described herein can be those such as, for example, shape memory polymers provided by Qualia Labs, INC.

Metal contacts (i.e. electrodes or electrode contacts) in the solenoidal lead which can make contact with a nerve and deliver pulses from the pulse generator can be constructed from a conductive metal, such as platinum, gold, and silver. In certain embodiments, the metal contacts are platinum. In embodiments, each contact has its own discrete lead in electrical communication with the pulse generator, allowing for discrete or synchronous operation of the contacts.

In certain aspects, the leads, contacts, or both can be encapsulated in the in the flexible polymer. The construct can be prefabricated in the sinusoidal fashion with the helical inner diameter matched to the average diameter of a nerve of a subject, for example the human sciatic nerve. Each contact of the solenoidal lead would expose itself to the nerve through a window in the solenoidal lead, with one contact per window.

The solenoidal lead can be in electrical connection with the pulse generator by way of a plurality of leads, wherein each one of the plurality of leads has an end in electrical connection with one electrode contact, and another opposing end that is in electrical communication with the pulse generator. Each of the plurality of leads can be constructed from a conductive metal, such as a platinum, gold, silver, and the like. In an embodiment, the leads are made of platinum.

The lead can be physically connected to the pulse generator by way of a cable, the cable comprising the plurality of leads. The cable can be a structure that is continuous with the pulse generator, the solenoidal lead, or both. In certain aspects, the pulse generator can detach from the cable (and therefore leads) at a detach point. In certain aspects, the solenoidal lead can detach from the cable (and therefore the leads) at a detach point. In certain aspects, the cable can detach from both the solenoidal lead and the pulse generator, or one or the other. The cable can have a sheath that encases the plurality of leads, for example a silicone or flexible polyethylene polymer.

Methods of using devices as described herein for peripheral neuromodulation can comprise delivering one or more pulses or a plurality of pulses to the nerve. Pulses as described herein can be sinusoidal waves at a kilohertz frequency stimulation from about 2 to about 100 kHz. Pulses as described herein can be kilohertz frequency pulses with a voltage range of about 0.25 to about 9 volts. Pulses as described herein can be sinusoidal waves at a kilohertz frequency stimulation from about 2 to about 10 kHz. Pulses as described herein can be kilohertz frequency pulses with a voltage range of about 0.25 to about 1 volts. Pulses as described herein can be sinusoidal waves at a kilohertz frequency stimulation from about 10 to about 90 kHz. Pulses as described herein can be kilohertz frequency pulses with a voltage range of about 1 to about 8 volts. Pulses as described herein can be sinusoidal waves at a kilohertz frequency stimulation from about 20 to about 80 kHz. Pulses as described herein can be kilohertz frequency pulses with a voltage range of about 2 to about 7 volts. Pulses as described herein can be sinusoidal waves at a kilohertz frequency stimulation from about 30 to about 70 kHz. Pulses as described herein can be kilohertz frequency pulses with a voltage range of about 3 to about 6 volts. Pulses as described herein can be sinusoidal waves at a kilohertz frequency stimulation from about 40 to about 60 kHz. Pulses as described herein can be kilohertz frequency pulses with a voltage range of about 4 to about 5 volts. Pulses as described herein can be sinusoidal waves at a kilohertz frequency stimulation from about 50 kHz. Pulses as described herein can be kilohertz frequency pulses with a voltage range of about 3 volts. Pulses as described herein can be sinusoidal waves at a kilohertz frequency stimulation from about 20 to about 30 kHz. Pulses as described herein can be kilohertz frequency pulses with a voltage range of about 1 to about 2 volts. Pulses as described herein can be sinusoidal waves at a kilohertz frequency stimulation from about 30 to about 40 kHz. Pulses as described herein can be kilohertz frequency pulses with a voltage range of about 2 to about 3 volts. Pulses as described herein can be sinusoidal waves at a kilohertz frequency stimulation from about 30 to about 40 kHz. Pulses as described herein can be kilohertz frequency pulses with a voltage range of about 3 to about 4 volts. Pulses as described herein can be sinusoidal waves at a kilohertz frequency stimulation from about 40 to about 50 kHz. Pulses as described herein can be kilohertz frequency pulses with a voltage range of about 4 to about 5 volts. Pulses as described herein can be sinusoidal waves at a kilohertz frequency stimulation from about 50 to about 60 kHz. Pulses as described herein can be kilohertz frequency pulses with a voltage range of about 5 to about 6 volts. Pulses as described herein can be sinusoidal waves at a kilohertz frequency stimulation from about 60 to about 70 kHz. Pulses as described herein can be kilohertz frequency pulses with a voltage range of about 6 to about 7 volts. Pulses as described herein can be sinusoidal waves at a kilohertz frequency stimulation from about 70 to about 80 kHz. Pulses as described herein can be kilohertz frequency pulses with a voltage range of about 7 to about 8 volts. Pulses as described herein can be sinusoidal waves at a kilohertz frequency stimulation from about 80 to about 90 kHz. Pulses as described herein can be kilohertz frequency pulses with a voltage range of about 8 to about 9 volts. Pulses as described herein can be sinusoidal waves at a kilohertz frequency stimulation from about 90 to about 100 kHz. In embodiments of the present disclosure, pulses as described herein consist of 50 kHz stimulation frequency at 3 volts.

The pulses can be varied to the patient needs, as a skilled artisan would surmise. In an embodiment, devices as described herein can deliver a nerve block with pulses comprising stimulation frequencies of about 50 kilohertz to about 70 kilohertz or greater. Nerve blocks as delivered by methods and devices as described herein can be rapidly reversible.

The pulse generator can be run continuously during the day, or can be adaptive and respond to physical activity or rest via a feedback loop from feedback device, such as an internal accelerometer. In an embodiment, therapy using methods and devices as described herein would comprise 50 kHz stimulation at 3 volts delivered during periods of perceived acceleration (when patient is awake and moving). For other painful conditions such as phantom leg pain, the pulse generator may deliver kilohertz frequency stimulation only during sleep hours, or when internal accelerometer senses rest during night hours.

Devices and methods herein can modulate symptoms of peripheral nerve dysfunction. Devices and methods as described herein can modulate pain or other perceived uncomfortable or otherwise noxious sensations by a subject. Devices and methods as described can be used for the treatment of sciatica, peripheral nerve compression pain, and chronic pain syndromes including but not limited to diabetic neuropathy, phantom leg pain, or dystonia. Devices and methods as described herein can block or scramble pain signals ascending from a peripheral nerve, or cancel the pain signal by method of descending block. In certain aspects, uncomfortable or otherwise noxious sensations can be, or can be similar to, symptoms of sciatica (electric shock-like discomfort running down leg, burning, throbbing, stabbing, crampy discomfort), diabetic neuropathy (itchy, burning, tingling, cooling, persistent discomfort), and phantom leg pain (shooting, stabbing, boring, squeezing, throbbing, burning pain. Pain when phantom limb feels as if it is being forced into an uncomfortable position, emotional stress). Other symptoms of peripheral nerve dysfunction that can be modulated by systems and devices as described herein can include painful dysesthesias resulting from conditions as described herein.

As described herein, devices and methods as described herein can deliver kilohertz electrostimulation to provide neuromodulation for a subject in need thereof. A subject in need thereof can be a patient experiencing one or more of sciatica, peripheral nerve compression pain, chronic pain syndromes (including but not limited to diabetic neuropathy, phantom leg pain, or dystonia), or perceived uncomfortable or noxious stimuli, acute or chronic. In embodiments according to the present disclosure, a subject in need thereof may be a subject experiencing symptoms of dorsal root ganglia (DRG) compression (for example a subject with one or more lateral disc herniation[s], or a subject with neuro-formainal stenosis from arthritis). In additional examples, a subject in need thereof may be a subject with nerve compression (for example fibular, tibular, sciatic) from tumors, arthritis, rheumatoid arthritis, peripheral cysts, post-operative scarring from orthopedic or peripheral vascular procedures, and the like.

In an embodiment, devices and methods as described herein can employ peripheral nerve modulation to block sciatic pain, as well as a sciatic neuromodulation device. The sciatic neuromodulation device is designed to encircle a human sciatic nerve with leads attached to stimulating pulse generator that can be implanted and is capable of delivering adjustable pulse electrostimulation to the nerve to block pain signaling. The proximal portion (solenoidal lead) that surrounds the nerve is made of an elastic polymer that can surgically be wrapped around the sciatic nerve. Within this polymer are a plurality of contacts that deliver electric pulses from the pulse generator directly to the nerve. The solenoid lead is attached to a cable that is connected to the pulse generator and implanted subcutaneously in the patient. The pulse generator is capable of delivering current that can be tuned externally. The electricity can be modulated with respect to its amperage, frequency, and duration of pulse. Pulses delivered from the device will serve to modulate sensory signals from the nerve to the spinal cord either by descending drive, ascending block, and/or a combination of both modalities. A skilled artisan will recognize that devices and methods as described herein are not limited to the sciatic nerve, and the device and methods could be expanded to any peripheral nerve that has been found to generate pain in a human. Furthermore, a skilled artisan will recognize that pulses delivered by systems and methods as described herein can be tuned specifically accordingly to the needs of that individual.

Adjustments (i.e. tuning) to the neurostimulation can be made by the user or physician through way of an application on a computer or portable computing device (for example, without intending to be limiting, a tablet personal computing device, smartphone, and the like) in wireless communication with the neurostimulator through a wireless communication protocol, for example Bluetooth®.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

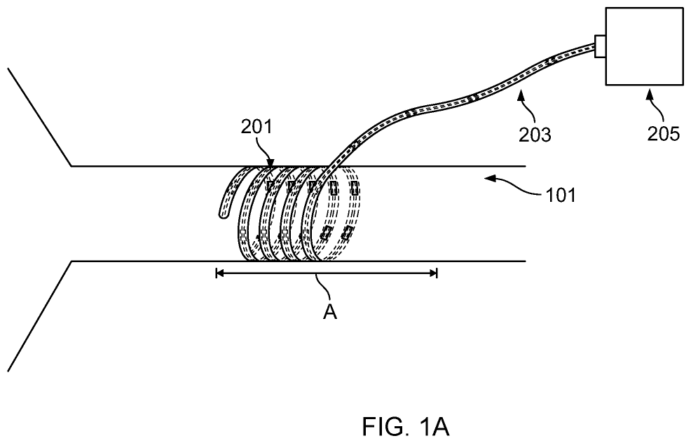

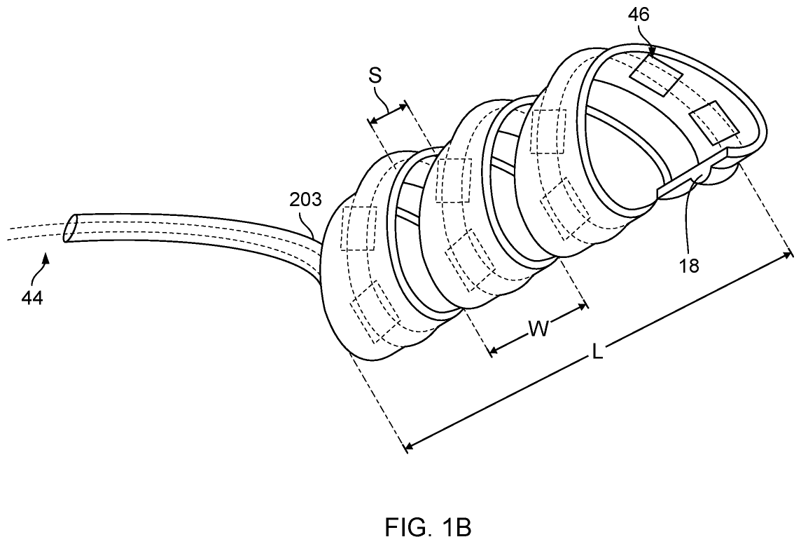

FIG. 1A depicts an embodiment of a peripheral nerve modulator as described herein. According to the embodiment depicted, the peripheral nerve modulator comprises a pulse generator 205 that is connected to a solenoid lead 201 by way of a cable 203, the solenoid lead being configured to wrap around and make contact with the outside circumference of a peripheral nerve 101 along a longitudinal axis of the peripheral nerve in the direction A. Electrical contact between the solenoidal lead and pulse generator is by the way of conductive leads housed in the cable 203, with each contact having its own discrete lead. It is to be understood that the cable 203 and polymeric material of the soldenoidal lead 201 can be of different materials.

In additional aspects of the peripheral nerve modulator, mainly that for the sciatic nerve, the sinusoidal cuff (i.e. solenoidal lead) would have the flexible capacity (due to the polymer used to encase the platinum-iridium contacts) between approximately 1-2 cm inner diameter for the sciatic nerve. Also, the coil loops would be configured to be approximately 0.5 cm apart between turns. There would be 4-5 complete turns around the nerve (spacing between each “turn” would be at least 0.5 cm). Each complete turn would have the circumferential contact as previously described.

FIG. 1B depicts an enlarged perspective view of another embodiment of the solenoidal lead 201. As can be seen from the enlarged perspective view, the length L of the solenoidal lead can comprise 3 turns around the nerve (not pictured; 4 turns and 5 turns in other aspects of the lead 201). The cable 203 can house leads 44 that connect the pulse generator (not pictured) to the contacts of the solenoidal lead 46. Each of the contacts 46 can have its own discrete lead 44 that connects the contact to the pulse generator. The turns of the solenoidal lead can have a distance S between them that can be about 0.5 cm in an embodiment. The solenoidal lead 201 can have a width W. Although a bulge 18 is shown in the polymer of the solenoidal lead that can house the leads, it is understood that the bulge is optional, and the soldenoidal lead can have an even thickness across the width W.

### Example 2

Introduction:

Chronic sciatica, resistant to medical and surgical therapies, affects approximately 2% of the world population over a lifetime. Pain management strategies often result in long-term opioid use, with a therapeutic profile resulting in dependence, dosage increases, and diminished benefit. A durable surgical cure is paramount, but complicated by the mixed-fiber nature of the lumbar plexus. Described herein is a pre-clinical study of sciatic neuromodulation, eliminating allodynic responses in a rat model of sciatic neuropathy with preservation of motor function.

Methods:

Lewis rats (n=5) had 2 mm PE-60 cuffs placed around their right sciatic nerve per validated neuropathy model, with silicon added and wires lengthened to ensure a proper fit. Distal to the cuff, a circumferential neurostimulator was implanted, with wires subcutaneously tunneled to a fixed head port. Prior to surgery, the rats received baseline von Frey testing, with objective responses recorded as paw-licks. At 1 and 2 weeks postoperatively, the animals underwent von-Frey testing of both paws pre-stimulation, during stimulation (50 kHz/3V/sinusoidal wave), and post stimulation. Paw-licks were tabulated as 50% withdrawal threshold (50% WD) with ANOVA analysis between phases.

Results:

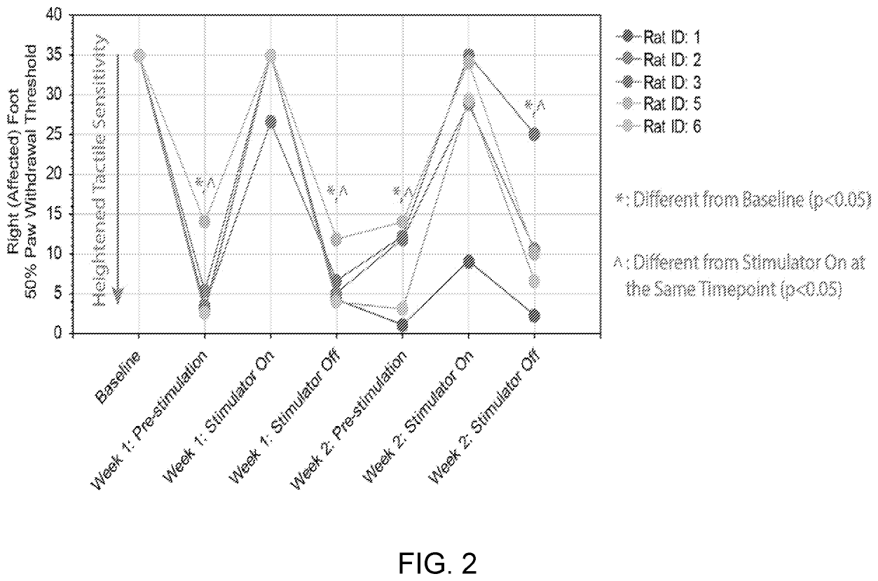

All 5 rats at baseline were out of range on von-Frey testing (50% WD=35). At week 1, the right paw threshold averages were 5.42 pre-stimulation (range 2.67-14.03), 33.31 during stimulation (range 26.57-35.00), and 6.40 post-stimulation (range 4.01-11.89). At week 2, the right paw thresholds were 8.47 pre-simulation (range 1.11-14.03), 27.31 during stimulation (range 9.12-35.00), and 10.97 post-stimulation (range 2.36-25.07). Stimulation restored limb sensitivity to near-baseline levels, and significantly differed from pre-stimulation and post-stimulation (p<0.05, see FIG. 2).

FIG. 2 illustrates data showing the 50% withdrawal threshold for the above subjects depending on whether the stimulator (configured for kilohertz stimulation) was turned on or off. The rodent model of neuropathy/allodynia employed in the present example was a right (right hindleg) sciatic nerve cuff to induce a painful neuropathy/allodynia in each animal. As shown in FIG. 2, at the prestimulation week 1 and week 2, the rats were withdrawing their paw to much lighter weight von Frey filaments compared to the baseline testing on the right paw prior to surgery. With the stimulator cony the paws nearly returned to their baseline. When the stimulator was turned off, they all nearly dropped down to the painful prestimulation allodynic response pattern.

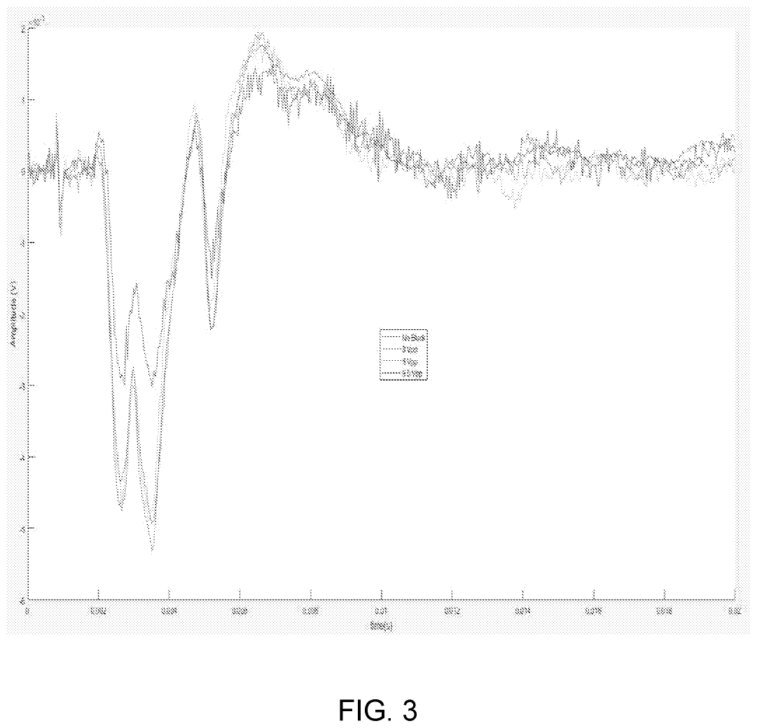

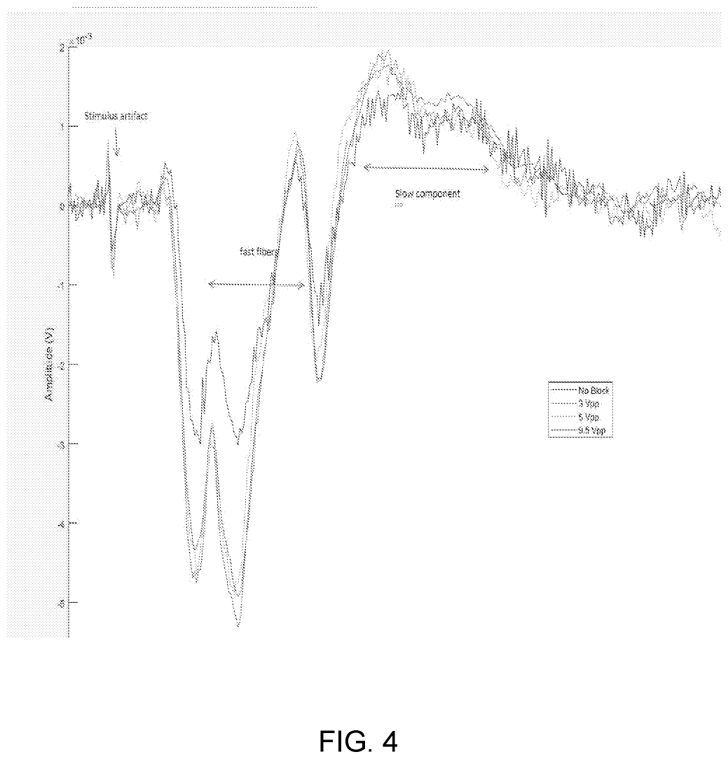

The plots of FIG. 3 and FIG. 4 show stimulated compound action potentials in a live rat sciatic nerve (average of 20 stimulations per line) with the bipolar stimulation cuff around the sciatic nerve and the recording cuff more distally around the tibial nerve (right side). The blocking cuff of the neurostimulator is placed in between the stimulation cuff and recording cuff, on the sciatic nerve. As the FIG. 3 and FIG. 4 legends state, the blue line is the AP with no block introduced. The other lines are with blocks applied, all at a frequency of 50 kHz and 3, 5, and 9.5 volts peak to peak. The fast fibers and slow fibers are marked on the graph (FIG. 4). Table 1 below shows the integrated values for the fast fiber and slow fiber areas under their curves on the amplitude-time plot. The integral value represents a % fiber activation. Therefore, as the table suggests, when the blocking cuff is activated at 50 kHz and 3 volts there is decreased activation of slow fibers when compared to baseline, with no decrease in fast fiber activation. These data suggest a fiber selective block at 50 kHz and 3 volts (unblocked area=0.1073, 50 kHz/3V area=0.1035), further suggesting approximately 3.54% of slow fibers are blocked with little to no block of fast fibers.

TABLE 1
Integrated Values for Fast and Slow Fiber Areas

| 50 kHz | Vpp | Fast valley I | Fast peak I | Slow I |
|---|---|---|---|---|
| | none | −.166.6 | −.0866 | .1073 |
| | 3 | −.1814 | −.0947 | .1035 |
| | 5 | −.1614 | −.0834 | .0950 |
| | 9.5 | −.099 | −.0519 | .0933 |

Conclusions:

Sciatic neuromodulation at kilohertz-level frequency produces selective nerve block in a rat model of peripheral neuropathy, where tactile allodynic responses to von Frey testing normalize in a rapidly reversible fashion. Additionally, visual inspection of rodent gait during stimulation indicates a mobile limb with no visual evidence of motor impairment, suggesting fiber-level selectivity.

Applications and Improved Patient Care:

An untold number of people around the word suffer from chronic, debilitating peripheral pain syndromes that lack a durable medical or surgical cure. The majority of these individuals resort to chronic opioid use or other temporary conservative measures to partially relieve their extremity pain. The pre-clinical study herein demonstrates that the application of kilohertz frequency neurostimulation to a validated sciatica model relieves allodynic pain responses via selective nerve fiber block, with visual improvement of gait. This model and these results of kilohertz stimulation can be employed in an implantable human modulation system to treat symptoms of or cure painful peripheral neuropathies.

Resources:
Patel Y A, Butera R J. Challenges associated with nerve conduction block using kilohertz electrical stimulation. J Neural Eng. 2018; 15(3):031002.
Mosconi T, Kruger L. Fixed-diameter polyethylene cuffs applied to the rat sciatic nerve induce a painful neuropathy: ultrastructural morphometric analysis of axonal alterations. Pain. 1996; 64(1):37-57.

### Example 3

Novel Kilohertz Frequency Neuromodulation for Fiber Selective Blockade of Sciatic Pain in a Rat Model

Introduction:

Pain management for chronic sciatica often results in long-term opioid use leading to dependence, dosage increases, and diminished benefit. It was discovered by the inventors that kilohertz (kHz) frequency modulation (50 kHz/3 Volts) of the sciatic nerve (SN), a novel stimulation paradigm described at length above, eliminates tactile allodynic responses in a validated rat model of sciatic neuropathy, with visual preservation of motor function. In the present example, the selective slow fiber (<5 m/s) inhibition observed with the 50 kHz/3V modulation is inspected and quantified.

Methods:

A Lewis rat was placed under general anesthesia and right sciatic nerve (SN) exposed. A dual-electrode stimulator cuff was implanted on the proximal SN, a recording cuff implanted around the tibial nerve distally (1.9 cm separation), and SN neuromodulator between the cuffs. Compound action potentials (CAPs) were elicited with charge-balanced 500 μA/0.1 ms biphasic pulses. Frequency-voltage combinations (30-100 kHz in 5 kHz increments at 3, 5, 7, and 9 V peak-to-peak, 15×4=60 trials) were applied to the SN during CAP induction. A trial comprised a trial of 50 kHz 3V stimulation with 5 CAPs before, 20 CAPs during, and 10 CAPs after neuromodulation. CAPs were recorded on an amplitude-time graph with integrals of peaks corresponding to specific fiber activation.

Results:

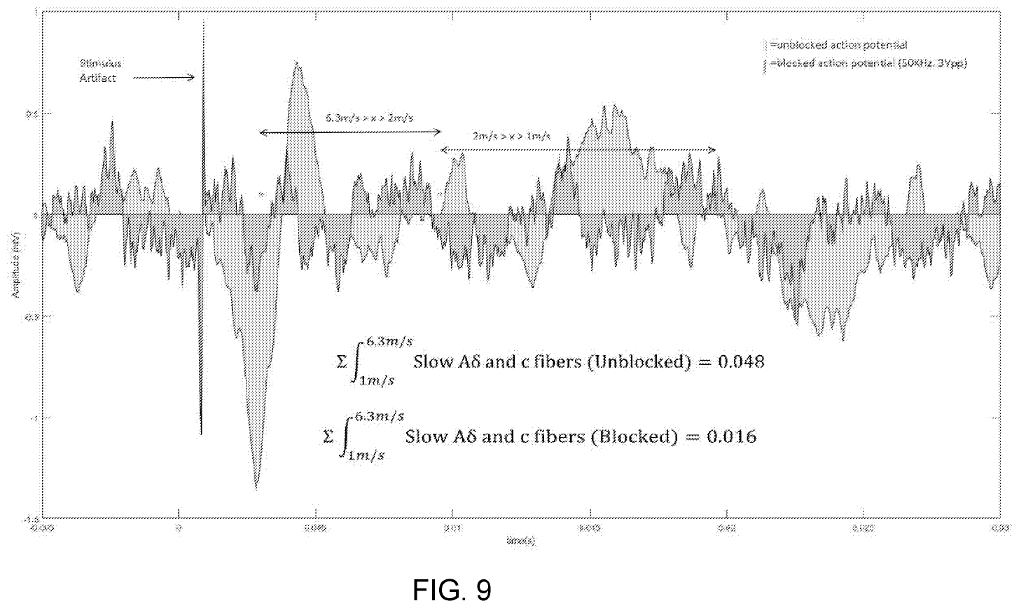

The integral of the <5 m/s combined peak before and during 50 kHz/3V modulation was 0.048 and 0.016, respectively, representing 67.0% slow fiber inactivation with preservation of muscle stimulus artifact indicating unblocked motor neurons. The integrals of the 4.7 m/s (slow Aδ fiber) and 1.3 m/s (c fiber) peaks before/during modulation were 0.023/0.007 and 0.025/0.009, representing 69.6% and 64.0% inactivation, respectively (FIG. 9).

Conclusions:

Sciatic neuromodulation at kilohertz frequency can produce rapidly reversible sensory nerve block in a rat model of peripheral neuropathy. The present example provides data that supports an electrophysiological explanation for the selective muting of downstream-source allodynic discomfort and upstream neuropathy with respect to the neuromodulator location observed in initial sciatica rat studies. The present example also offers support of the feasibility of a pulse generator-sciatic system for durable treatment of painful neuropathy in humans.

### Example 4

Fiber-Selective Peripheral Neuromodulation for Treatment of Sciatic Peripheral Neuropathy in a Rat Model

Introduction:

Chronic sciatica resistant to medical and surgical therapies affects approximately 2% of the world population over a lifetime. Pain management strategies often result in long-term opioid use, with a therapeutic profile resulting in dependence, dosage increases, and diminished benefit. A durable surgical cure is paramount, but complicated by the mixed-fiber nature of the lumbar plexus. Our group describes a pre-clinical study of sciatic neuromodulation, eliminating allodynic responses in a rat model of sciatic neuropathy with preservation of motor function.

Methods:

Lewis rats (n=5) had 2 mm PE-60 cuffs placed around their right sciatic nerve per validated neuropathy model. Distal to the cuff, a circumferential neurostimulator was implanted, with wires subcutaneously tunneled to a fixed head port. Prior to surgery, the rats received baseline von-Frey testing, with objective responses recorded as paw-licks. At 1 and 2 weeks postoperatively, the animals underwent von-Frey testing of both paws pre-stimulation, during stimulation (50 kHz/3V/sinusoidal wave), and post stimulation. Paw-licks were tabulated as 50% withdrawal threshold (50% WD) with ANOVA analysis between phases.

Results:

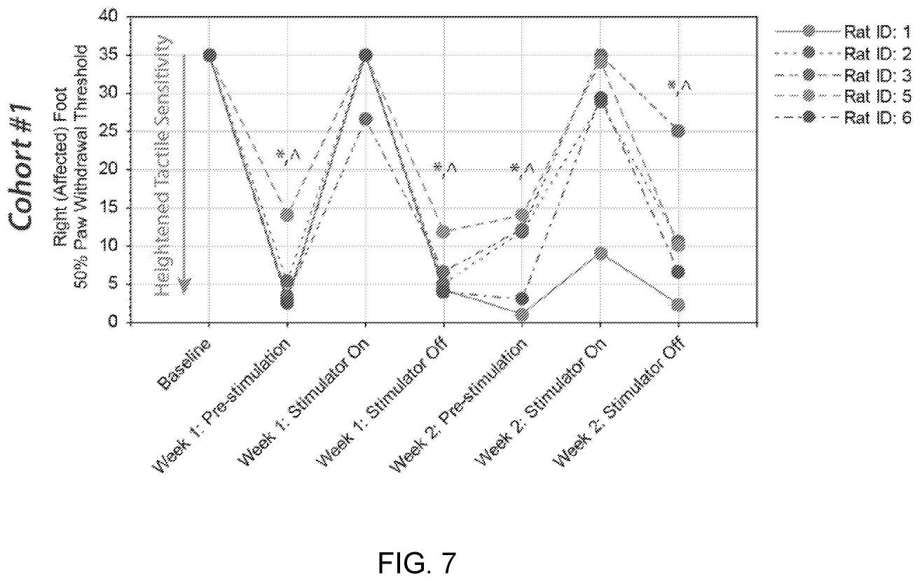

All 5 rats at baseline were out of range on von-Frey testing (50% WD=35). At week 1, the right paw threshold averages were 5.42 pre-stimulation (range 2.67-14.03), 33.31 during stimulation (range 26.57-35.00), and 6.40 post-stimulation (range 4.01-11.89). At week 2, the right paw thresholds were 8.47 pre-simulation (range 1.11-14.03), 27.31 during stimulation (range 9.12-35.00), and 10.97 post-stimulation (range 2.36-25.07). Stimulation restored limb sensitivity to near-baseline levels, and significantly differed from pre-stimulation and post-stimulation (p<0.05, see FIGS. 2 and 7).

Conclusions:

Sciatic neuromodulation at kilohertz-level frequency can produce selective nerve block in a rat model of peripheral neuropathy, where tactile allodynic responses to von Frey testing normalize in a rapidly reversible fashion. Additionally, visual inspection of rodent gait during stimulation indicates a mobile limb with no visual evidence of motor impairment, suggesting fiber-level selectivity.

Keywords:

Sciatic Neuromodulation, Von Frey Testing, Kilohertz Frequency Stimulation, Peripheral Neuropathy, Opioid Epidemic, Phantom Limb Pain, Diabetic Neuropathy

How Will Such Research Improve Patient Care:

It is believed that millions of people around the word suffer from chronic and debilitating peripheral pain syndromes that lack a durable medical or surgical cure. The majority of these individuals resort to chronic opioid use or other temporary measures to partially relieve their extremity pain. The present example demonstrates that the application of kilohertz frequency neurostimulation to a validated sciatica model can relieve allodynic pain responses via selective nerve fiber block, with visual improvement of gait. This model and research opens the door for development of an implantable human modulation system to cure painful peripheral neuropathies.

### RESOURCES

Patel Y A, Butera R J. Challenges associated with nerve conduction block using kilohertz electrical stimulation. J Neural Eng. 2018; 15(3):031002.
Mosconi T, Kruger L. Fixed-diameter polyethylene cuffs applied to the rat sciatic nerve induce a painful neuropathy: ultrastructural morphometric analysis of axonal alterations. Pain. 1996; 64(1):37-57.

### Example 5

Chronic sciatica resistant to medical and surgical therapies affects approximately 2% of the world population over a lifetime. Up to 53% of individuals undergoing laminectomy with discectomy have sciatic pain after 4 years, with 25% of relapse after a 2 year period of relief. The results of the SPORT trial show that 32%/36% of operative/non-operative patients at 2 years were LESS than “somewhat satisfied” with back/leg pain, and there is currently no durable surgical solution.

Chronic sciatica resistant to medical and surgical therapies affects approximately 2% of the world population over a lifetime. Up to 53% of individuals undergoing laminectomy with discectomy have sciatic pain after 4 years, with 25% of relapse after a 2 year period of relief. The results of the SPORT trial show that 32%/36% of operative/non-operative patients at 2 years were LESS than “somewhat satisfied” with back/leg pain, and there is currently no durable surgical solution.

It is believed that millions of people around the world suffer from symptoms of chronic, debilitating peripheral pain syndromes that lack a cure. Many resort to long-term opioid use for symptom relief.

Kilohertz electrical modulation (KEM) of central and peripheral nerves, as described herein, allows rapid, reversible, and focal conduction block. There are many potential benefits of peripheral neuromodulation, including a higher quality nerve interface and a palatable surgical risk profile vs. traditional spinal cord stimulation. Initial work of Yogi Patel on frog and rat sciatic nerves suggests fiber-selective inhibition of nerve compound action potentials (CAPs) above 50 KHz/1 mA. It is noted, however, that the work of Patel did not perform experiments in live animals with intact (injured or non-injured) nervous systems to assess behavior. It was not expected that the observations of Patel would apply to other experimental or clinical systems, especially given the variables or factors involved (for example electrode geometry, material, nerve thickness, etc). Patel's data was also based on frequency-amperage combinations (the work herein is utilizes, in aspects, frequency-voltage combinations) which was done to provide an electrophysiologic basis for the behavioral results that were observed.

In aspects of the present disclosure, kilohertz frequency sciatic neuromodulation (50 KHz/3V peak-to-peak sinusoidal wave) can reduce signs of pain-related behavior. In further aspects of the present disclosure, kilohertz frequency sciatic neuromodulation (50 KHz/3V) will selectively block slow fiber (<7 m/s) nerve impulses during CAP recording with preservation of muscle stimulus artifact indicating unblocked motor neurons.

Such aspects can be validated with chronic and acute surgery, for example, by the use of von-Frey testing in rats with sciatic nerve compression. Measures of success of aspects of the present disclosure can be assessed, for example, by assessing a reduction of paw-lick and selective reduction of slow fiber peak integrals on a CAP amplitude-time graph.

There are potential obstacles to successfully implementation of such strategies, including the mixed fiber nature of the sciatic nerve; lack of inherent somatotopy; and an upstream pain generator.

In an embodiment according to the present example, two cohorts of Lewis rats (1st cohort, acute, N=5, 2^{nd} cohort, chronic, N=6) were used to demonstrate aspects of the present disclosure.

2 mm PE-60 cuffs with an inner diameter of 0.03″ were placed around the right sciatic nerve. Distal to the injury cuff, a circumferential neurostimulator (dual electrode 1st cohort/Microprobe™ dual electrode 2^{nd} cohort) was implanted with wires subcutaneously tunneled to a fixed head port. The “1st Cohort” and “2^{nd} Cohort” were both chronic surgery cohorts. These animals underwent implantation of injury cuff upstream of the sciatic cuff (was a cuff with an inner diameter of 0.03 inches/1 mm). In the first cohort chronic surgery sciatic compression experiment, a cuff that was altered that was provided from Virginia tech, in the 2nd cohort chronic surgery sciatic compression experiment, a cuff purchased from Microprobes was used (dual hook electrode (platinum-iridium hooks spaced 1 mm apart, inner diameter adjustable, but about 1 mm for the hooks)). The ‘acute experiment’ as described in the entirety of the disclosure herein relates to a single rat study (n=1) where the animal was under anesthesia and CAPs were elicited in the nerve before-during-after sciatic neuromodulation at 50 kHz and 3 volts. Von-Frey testing was performed at baseline, with weekly testing of both paws pre-, during, and post-KEM for 4 weeks. Paw-licks were tabulated as 50% WD with ANOVA between phases.

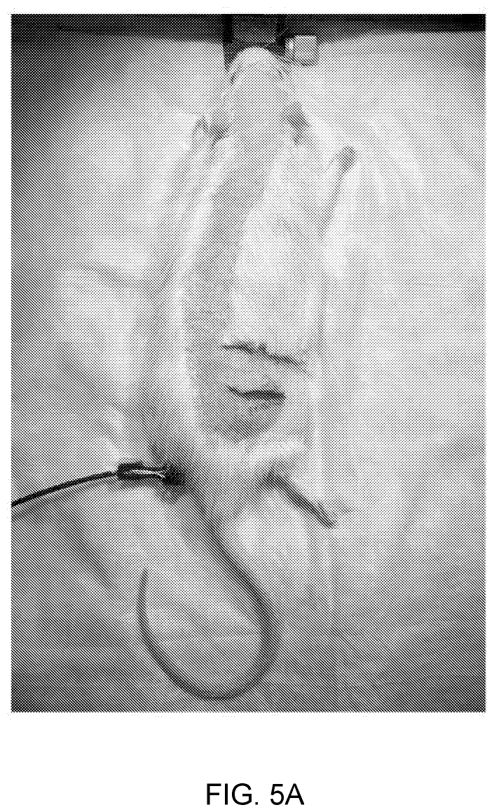

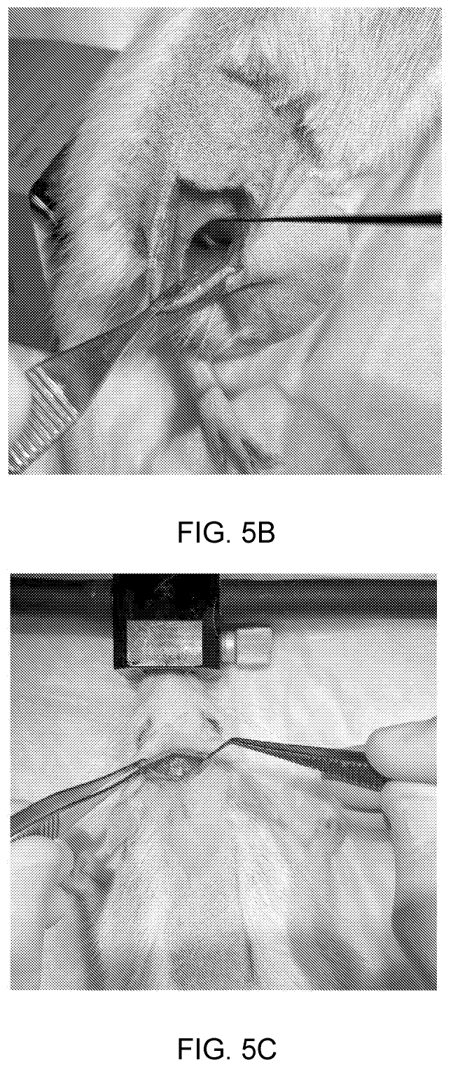

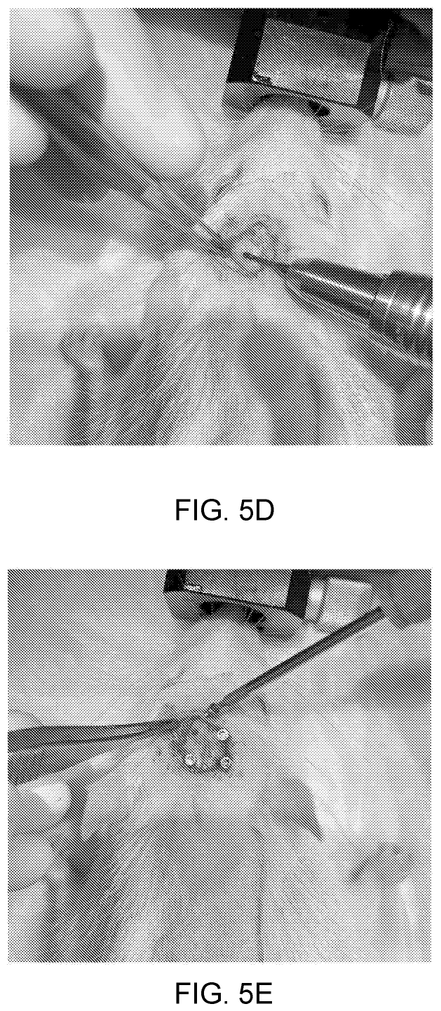

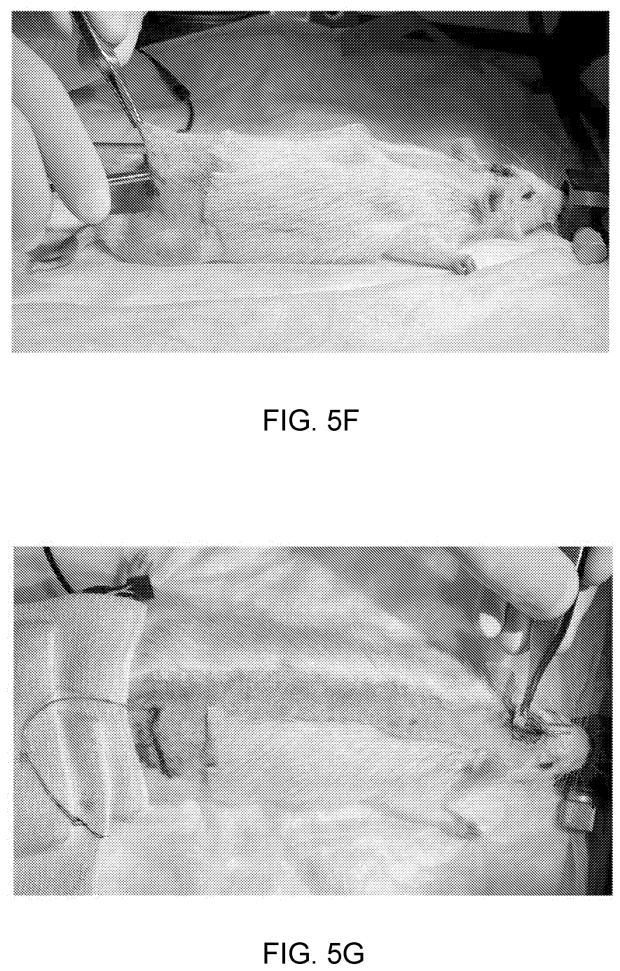

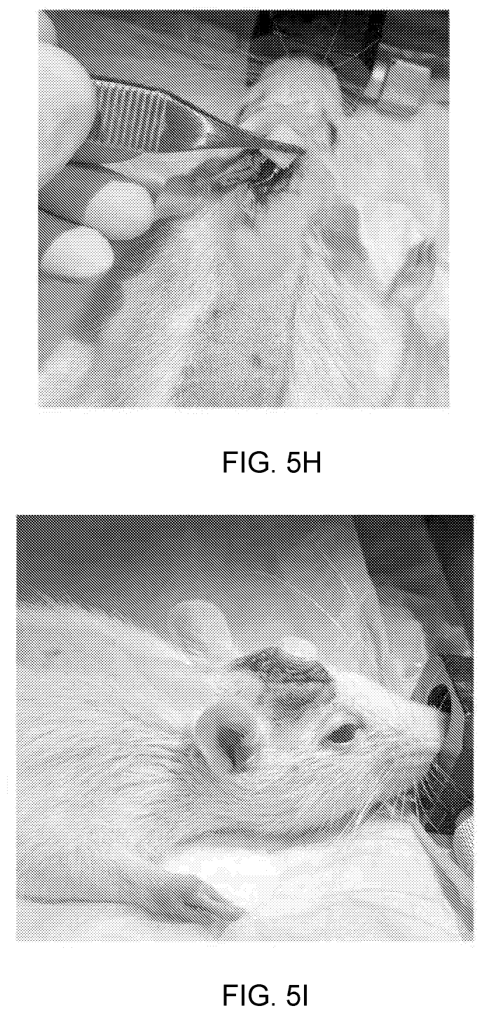

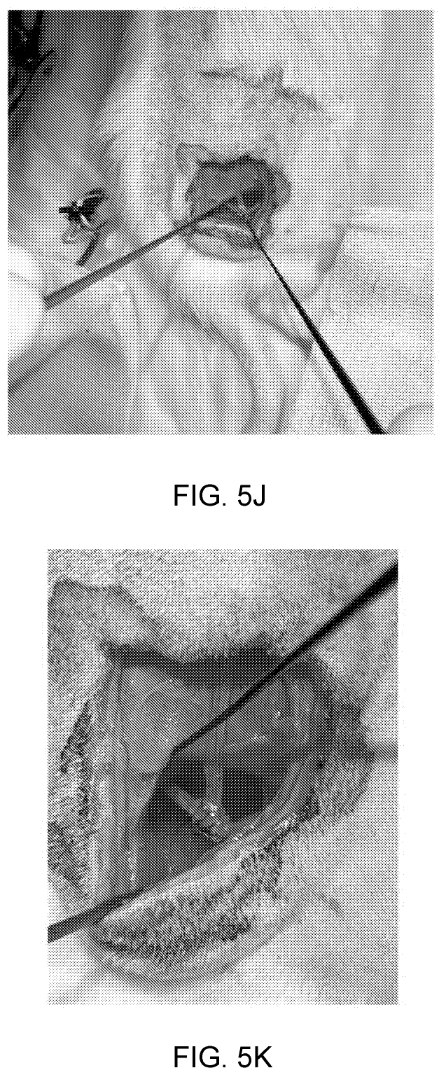

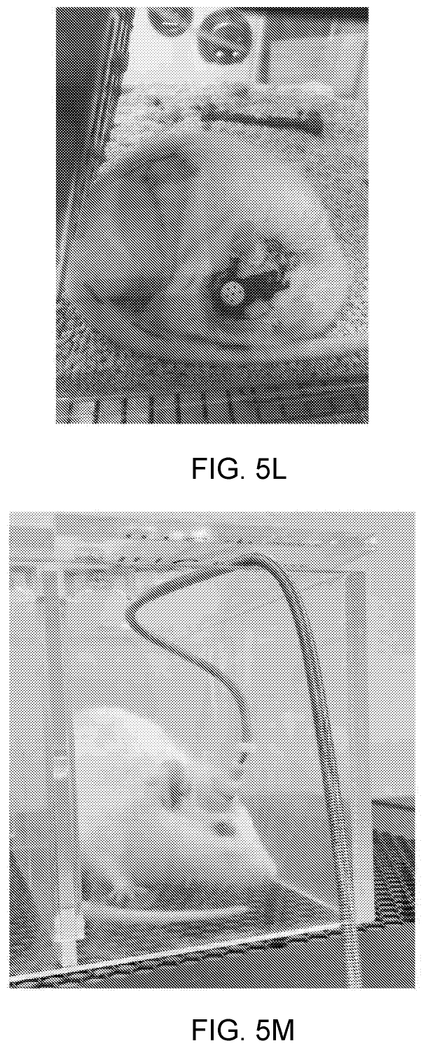

FIGS. 5A-5M are photographs showing aspects of embodiments of the present disclosure. FIG. 5A shows the initial incision across the right hindquarters to gain access to the right sciatic nerve of the rat. FIG. 5B illustrates separating the hindmuscles and subcutaneous anatomical structures in order to expose the sciatic nerve. FIG. 5C shows an incision made to gain access to the skull for head port placement. FIGS. 5D and 5E show burr hole drilling and screw fixation for secure head port placement. FIGS. 5F and 5G show subcutaneous tunneling for threading of the subcutaneous wires that provide a connection between the neurostimulator and head port. FIGS. 5H and 51 show head port placement, and FIGS. 5J and 5K show neurostimulator placement, and the placement of the cuff (FIG. 5K). FIGS. 5L and 5M show rodent recovery from surgery and recording leads from the head port.

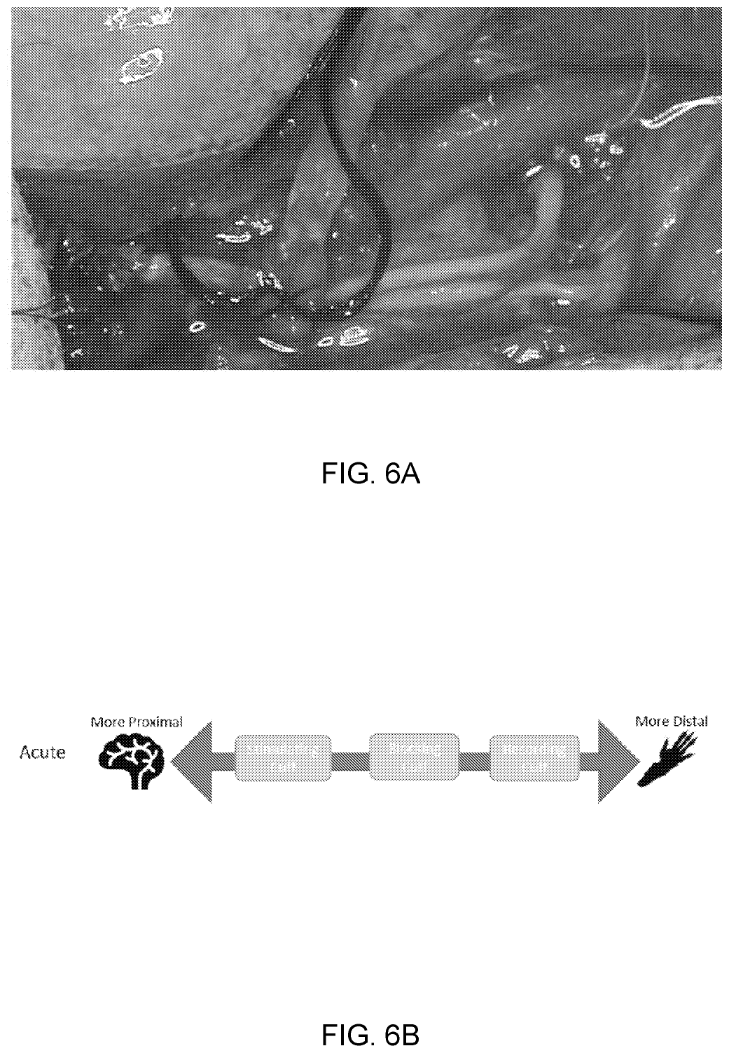

For acute surgery, a Lewis rat was used. Dual electrode stimulator cuff implanted on proximal sciatic nerve, recording cuff around the tibial nerve distally, and neuromodulator cuff between. As can be seen in FIG. 6A, shows the acute nerve crush injury with cuff placement and FIG. 6B is a graphic that illustrates the relative placement of the stimulating cuff, blocking cuff, recording cuff relative to the proximal and distal portions of the nerve.

FIG. 6A is from the ‘acute surgery’—there was no crush injury in the acute experiment. The FIG. 5A-5M pictures are representative of the ‘Chronic surgery methods’ as described herein. The chronic surgery was the surgery and experiment with the upstream compression cuff to cause injury. To describe this further, a similar diagram was presented for the chronic surgery as in FIG. 6B for the acute surgery, near the brain side (proximal), would be the compression injury cuff, and closer to the foot side (distal) would be the chronically implanted nerve stimulator cuff.

Compound action potentials (CAPs) were elicited with charge-balanced 500 μA/0.1 ms biphasic pulses. Frequency-voltage combination (30-100 kHz in 5 kHz increments at 3, 5, 7, and 9 V peak to peak) were applied to the sciatic nerve during CAP induction. 5 CAPs unblocked: 20 during modulation, CAPS were recorded on an amplitude-time graph.

Results

FIG. 7 demonstrates data of a Von Frey nociception assay in a first cohort (N=5) of an acute rodent model of neuropathy/allodynia with and without kilohertz stimulation. All rats baseline out of range (50% WD=35). Week 1 demonstrated a pre-stim average of 5.42 (2.67-14.0), stim average of 33.3 (26.6-35.0), and post-stim average of 6.40 (4.01-11.9). Week 2 demonstrated a pre-stim average of 8.47 (1.11-14.0), stim average of 27.3 (9.12-35.0), and post-stim average of 11 (2.36-25.1).

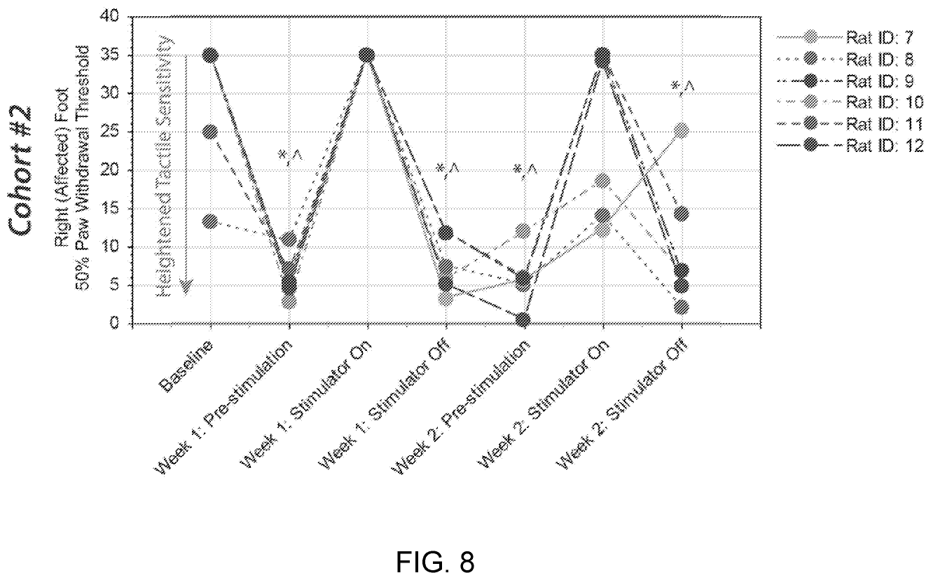

FIG. 8 demonstrates data of a Von Frey nociception assay in a second cohort (N=6) of rodent model of a chronic neuropathy/allodynia with and without kilohertz stimulation. All rats baseline out of range (50% WD=29.7). Week 1 demonstrated a pre-stim average of 5.98 (2.83-10.7), stim average of 35, and post-stim average of 7.51 (3.18-11.9). Week 2 demonstrated a pre-stim average of 5.75 (0.38-11.9), stim average of 24.8 (12.2-35.0), and post-stim average of 9.95 (2.00-25.1).

FIG. 9 is an amplitude-time graph showing compound action potentials (CAPs) following sciatic nerve stimulation with integrals of peaks corresponding to specific fiber activation. Overall, 67.0% slow-Aδ/c-fiber inactivation with block at 50 KHz/3 Vpp. Integrals of the 4.7 m/s (slow Aδ fiber) and 1.3 m/s (c-fiber) peaks before/during modulation were 0.023/0.007 and 0.025/0.009, representing 69.6% and 64.0% inactivation, respectively

Conclusions

Sciatic neuromodulation at kilohertz-level frequency can produce a selective nerve block in the rat model of peripheral nerve compression. Results demonstrate that tactile allodynic responses to von-Frey testing normalize in a rapidly reversible fashion. Visual inspection of rodent gait during stimulation (see example 6 below) indicates a mobile limb with improvement in limb cadence and paw contact, suggesting fiber-level selectivity. Acute experiments as described herein provide electrophysiological insight for the selective muting of downstream-source discomfort and upstream compression and proof of concept for an implantable pulse-generator system for durable treatment of painful neuropathy in humans.

It is noted that KHFAC (kilohertz frequency alternating currents) as described herein doesn't cause a propagating signal after the onset response. The nerve may be sensitized through compression injury, causing it to have a lower threshold to trigger an action potential. However, if these action potentials are blocked before they reach the central nervous system it may not matter how low the threshold is, because the action potentials are blocked.

### Example 6

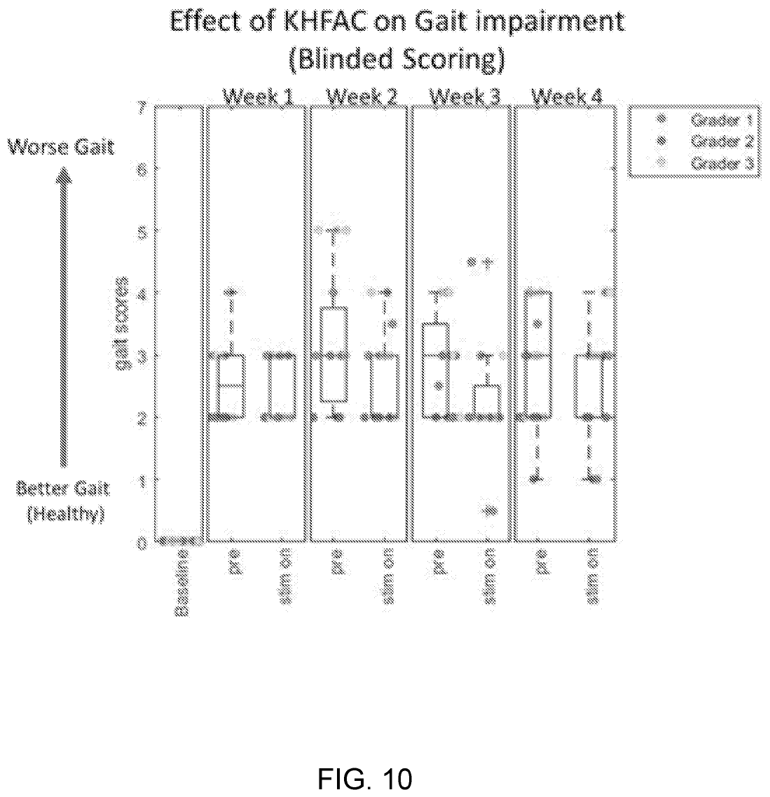

FIG. 10 illustrates plots of rank-order gait data of rat gait (following sciatic crush surgery) from videos of gait of cohort 2 (N=6) as described in the examples above with and without stimulation (stimulation was a continuous 50 kHz 3 Vpp AC) scored by blinded researchers. Using the following scale below, researchers were given randomly named videos of rats walking across the screen and asked to grade the quality of their gait.
0: Normal gait
1: Mild limping, guarding
2: No toe spread on the right foot
3: Not weight bearing, foot placed off to the side of the body. the foot is typically turned to the side.
4: Barely touching foot down, hopping
5: at least 1 cycle of 3 legged gait (1 step where the right foot didn't touch the ground)
6: complete 3 legged gait (right foot never touches the ground)
7: Not walking

The graded results show worse gait than baseline for all points after surgery, which is expected. Interestingly, there is no observed statistically significant difference between trials where the stimulator is on vs. off. This may indicate that the nerve block is not blocking motor nerves, which is beneficial for any clinically relevant treatment. The results are baseline before surgery, then at 1, 2, 3, and 4 week post-op with and without stimulation (labeled ‘stim on’ and ‘pre’, respectively). These are the same animals as cohort 2 (n=6), with 2 videos of each rat in each condition. Stimulation was a continuous 50 kHz 3 Vpp AC.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1) A peripheral neurostimulator, comprising:
a neuromodulator comprising a power source and an electric pulse generator;
a solenoidal lead comprising a flexible polymer and a plurality of metal contacts,
wherein each of the plurality of metal contacts are in electrical communication with the neuromodulator through a contact lead,
wherein none of the metal contacts shares a contact lead with another metal contact,
wherein the metal contacts are configured to make contact with a peripheral nerve of a subject from an inner surface of the solenoidal lead, the metal contacts extending equidistantly the length of a longitudinal axis of the solenoidal lead; and
a cable physically connecting the solenoidal lead to the neuromodulator, the cable comprising a plurality of contact leads providing electrical communication between the metal contacts of the solenoidal lead and the neurostimulator.

2) The peripheral neurostimulator of claim 1, wherein the solenoidal lead is helically shaped.

3) The peripheral neurostimulator of claim 1, wherein the solenoidal lead is configured to wrap around a peripheral nerve prior to implantation in a subject.

4) The peripheral neurostimulator of claim 1, wherein the solenoidal lead comprises about 3 turns around the nerve or more.

5-6) (canceled)

7) The peripheral neurostimulator of claim 1, where in the plurality of contact leads and metal contacts are platinum.

8) The neurostimulator of claim 1, wherein the neuromodulator is configured for kilohertz stimulation.

9) A method of treatment of a disease or symptoms of a disease in a subject, comprising:
providing a subject in need thereof;
implanting the neurostimulator of claim 1 into a subject in need thereof; and
delivering kilohertz stimulation to the subject in need thereof from the neurostimulator.

10) The method of claim 9, wherein the kilohertz stimulation is about 2 kilohertz to about 100 kilohertz.

11) (canceled)

12) The method of claim 9, wherein the subject in need thereof is a subject having or having symptoms of one or more of sciatica, peripheral nerve compression pain, chronic pain syndromes diabetic neuropathy, phantom leg pain, dystonia, noxious stimuli, or uncomfortable stimuli.

13) The method of claim 9, wherein the kilohertz stimulation has a voltage of about 0.25 volts to about 9 volts.

14) (canceled)

15) The method of claim 9, wherein the neurostimulator further comprises an accelerometer, and the kilohertz stimulation is delivered in response to a signal from the accelerometer.

16) The neurostimulator of claim 1, wherein the neurostimulator further comprises an accelerometer.

17) A solenoidal lead comprising:
a flexible polymer, a plurality of contact leads, and a plurality of metal contacts, the plurality of contact leads and metal contacts partially encased in the flexible polymer,
wherein each of the plurality of metal contacts are in contact with a contact lead,
wherein none of the metal contacts shares a contact lead with another metal contact, and
wherein a surface of each of the metal contacts is configured to make contact with a peripheral nerve of a subject from an inner surface of the solenoidal lead, the metal contacts extending equidistantly the length of a longitudinal axis of the solenoidal lead.

18) The solenoidal lead of claim 17, further comprising a cable encasing portions of the contact leads not encased in the flexible polymer.

19) The solenoidal lead of claim 17, wherein the solenoidal lead is helically shaped.

20) The solenoidal lead of claim 17, wherein the solenoidal lead is configured to wrap around a peripheral nerve prior to implantation in a subject.

21) The solenoidal lead of claim 17, wherein the solenoidal lead comprises about 3 turns around the nerve or more.

22) (canceled)

23) The solenoidal lead of claim 17, wherein the solenoidal lead comprises at least 4 metal contacts per turn of the solenoidal lead.

24) The solenoidal lead of claim 17, wherein the plurality of contact leads and metal contacts are platinum or platinum-iridium.

25-26)

27) The method of claim 9, wherein the implanting comprises placing a peripheral nerve of the subject inside the pre-configured helix of the solenoidal lead.
